SEQUENCE LISTING (28 sequences)

SEQ ID NO: 1 - protein, 14 aa, Artificial Sequence
  Description of Artificial Sequence Synthetic peptide
RGDACXGDSGGPXV

SEQ ID NO: 2 - protein, 23 aa, Artificial Sequence
  Description of Artificial Sequence Synthetic peptide
AGYKPDEGKRGDACXGDSGGPXV

SEQ ID NO: 3 - protein, 23 aa, Artificial Sequence
  Description of Artificial Sequence Synthetic peptide
AGYKPDEGKRGDACEGDSGGPFV

SEQ ID NO: 4 - protein, 14 aa, Artificial Sequence
  Description of Artificial Sequence Synthetic peptide
RGDAXXGDSGGPXV

SEQ ID NO: 5 - protein, 23 aa, Artificial Sequence
  Description of Artificial Sequence Synthetic peptide
AGYKPDEGKRGDAXXGDSGGPXV

SEQ ID NO: 6 - protein, 23 aa, Artificial Sequence
  Description of Artificial Sequence Synthetic peptide
AGYKPDEGKRGDACEGDSGGPFV

SEQ ID NO: 7 - protein, 10 aa, Artificial Sequence
  Description of Artificial Sequence Synthetic peptide
EGKRGDACEG

SEQ ID NO: 8 - protein, 33 aa, Artificial Sequence
  Description of Artificial Sequence Synthetic polypeptide
AGYKPDEGKRGDACXGDSGGPXVMKSPFNNRWY

SEQ ID NO: 9 - protein, 33 aa, Artificial Sequence
  Description of Artificial Sequence Synthetic polypeptide
AGYKPDEGKRGDACEGDSGGPFVMKSPFNNRWY

SEQ ID NO: 10 - protein, 12 aa, Artificial Sequence
  Description of Artificial Sequence Synthetic peptide
DACXGDSGGPXV

SEQ ID NO: 11 - protein, 14 aa, Artificial Sequence
  Description of Artificial Sequence Synthetic peptide
RGDAXEGDSGGPFV

SEQ ID NO: 12 - protein, 23 aa, Artificial Sequence
  Description of Artificial Sequence Synthetic peptide
AGYKPDEGKRGDACXGDSGGPXV

SEQ ID NO: 13 - protein, 12 aa, Artificial Sequence
  Description of Artificial Sequence Synthetic peptide
DXCXGDSGGPXV

SEQ ID NO: 14 - protein, 10 aa, Artificial Sequence
  Description of Artificial Sequence Synthetic peptide
CEGDSGGPFV

SEQ ID NO: 15 - protein, 10 aa, Artificial Sequence
  Description of Artificial Sequence Synthetic peptide
CXGDSGGPXV

SEQ ID NO: 16 - protein, 4 aa, Artificial Sequence
  Description of Artificial Sequence Synthetic peptide
RGDA

SEQ ID NO: 17 - protein, 14 aa, Artificial Sequence
  Description of Artificial Sequence Synthetic peptide
RGDACEGDSGGPFV

SEQ ID NO: 18 - protein, 33 aa, Artificial Sequence
  Description of Artificial Sequence Synthetic polypeptide
DNMFCAGYKPDEGKRGDACEGDSGGPFVMKSPF

SEQ ID NO: 19 - protein, 33 aa, Artificial Sequence
  Description of Artificial Sequence Synthetic polypeptide
DNMFCAGYKPDEGKRGDACXGDSGGPXVMKSPF

SEQ ID NO: 20 - protein, 23 aa, Artificial Sequence
  Description of Artificial Sequence Synthetic peptide
AGYKPDEGKRGDAXEGDSGGPFV

SEQ ID NO: 21 - protein, 23 aa, Artificial Sequence
  Description of Artificial Sequence Synthetic peptide
AGYKPDEGKRGDAAEGDSGGPFV

SEQ ID NO: 22 - protein, 23 aa, Artificial Sequence
  Description of Artificial Sequence Synthetic peptide
AGYKPDEGKRGDAAEGDSGGPFV

SEQ ID NO: 23 - protein, 33 aa, Artificial Sequence
  Description of Artificial Sequence Synthetic polypeptide
DNMFXAGYKPDEGKRGDAXEGDSGGPFVMKSPF

SEQ ID NO: 24 - protein, 33 aa, Artificial Sequence
  Description of Artificial Sequence Synthetic polypeptide
DNMFXAGYKPDEGKRGDAXXGDSGGPXVMKSPF

SEQ ID NO: 25 - protein, 33 aa, Artificial Sequence
  Description of Artificial Sequence Synthetic polypeptide
DNMFAAGYKPDEGKRGDAAEGDSGGPFVMKSPF

SEQ ID NO: 26 - protein, 33 aa, Artificial Sequence; identical to SEQ ID NO: 25
  Description of Artificial Sequence Synthetic polypeptide

SEQ ID NO: 27 - protein, 33 aa, Artificial Sequence; identical to SEQ ID NO: 9
  Description of Artificial Sequence Synthetic polypeptide

SEQ ID NO: 28 - protein, 23 aa, Artificial Sequence
  Description of Artificial Sequence Synthetic peptide
AGYKPDEGKRGDASEGDSGGPFV

(19) United States
(12) Patent Application Publication
(10) Pub. No.: US 2011/0105400 A1
(43) Pub. Date: May 5, 2011

(54) METHODS FOR TREATING ACUTE MYOCARDIAL INFARCTION

(75) Inventors: Randolph C. Steer, Rancho Mirage, CA (US); Michael R. Sheller, Tempe, AZ (US); John D. Wagstaff, Phoenix, AZ (US)

(73) Assignee: OrthoLogic Corp.

(21) Appl. No.: 12/934,585

(22) PCT Filed: Mar. 26, 2009
(86) PCT No.: PCT/US09/01954

§ 371 Date: Dec. 20, 2010

Related U.S. Application Data
(60) Provisional application No. 61/070,837, filed on Mar. 26, 2008.
(60) Provisional application No. 61/137,953, filed on Aug. 5, 2008.

(51) Int. Cl. A61K 38/17; A61K 38/10; A61P 9/10

(52) U.S. Cl. 514/16.4

(57) ABSTRACT

The present invention includes methods of treating acute myocardial infarction in a subject, comprising administering to the subject a therapeutically effective amount of a non-proteolytically activated thrombin receptor agonist.

### RELATED APPLICATIONS

This application claims the benefit of U.S. Provisional Application No. 61/070,837, filed on Mar. 26, 2008, and U.S. Provisional Application No. 61/137,953, filed on Aug. 5, 2008.

The entire teachings of the above applications are incorporated herein by reference.

### BACKGROUND OF THE INVENTION

Myocardial infarction (MI) is the leading cause of death in the many developed countries including the United States. The World Health Organization (WHO) has estimated that 13 percent of deaths worldwide are related to ischemic heart conditions ranging from silent ischemia to acute MI (AMI). Generally, AMI is caused by occlusion of a coronary artery typically resulting from atherosclerosis or embolism leading to a severe oxygen shortage (ischemia) that causes rapid cellular damage, or potentially death, of an area of the heart in which the blood supply is interrupted. AMI can be also caused by other ischemic events resulting from coronary spasm, anemia, arrhythmias, hypertension, hypotension or cardiac arrest. In some cases, percutaneous coronary intervention (PCI), in-stent thrombosis or coronary artery bypass graft (CABG) surgery are also known to cause AMI.

A number of different risk factors for heart disease leading to AMI include smoking, high blood pressure (hypertension), obesity, diabetes, a diet high in fat, and high blood cholesterol levels, particularly a high low-density lipoprotein (LDL) value combined with a low high-density lipoprotein (HDL) level. Other factors include gender, with males at higher risk, age, as well as genetic predisposition.

The onset of AMI is often characterized by angina pectoris (chest pain) that may radiate to the left arm, neck or epigastrium, and sometimes simulates the sensation of acute indigestion or gallbladder attack. The patient usually becomes short of breath, sweaty, nauseous, and/or faint. Typical signs are tachycardia, a barely perceptible pulse, low blood pressure, an elevated temperature, cardiac arrhythmia, and irregular electrocardiogram (EKG/ECG) evidencing an elevated ST segment and Q wave. Clinical tests may also indicate an increased erythrocyte sedimentation rate, leukocytosis, and elevated levels of serum enzymes (biochemical markers) such as creatine kinase (CK-MB), lactic dehydrogenase, glutamic-oxaloacetic transaminase, and troponins I, C and T. If two of the three typical manifestations (i.e., ischemic chest pain, irregular ECG signs, and elevated serum enzyme levels; also known as “WHO criteria”) are observed, a patient is diagnosed with AMI (Gillum et al., Am. Heart. J. (1984) 108:150-8; Tunstall-Pedoe et al., Circulation, 90:583-612 (1994)). Nevertheless, the current trend is to give more importance to elevated biochemical markers such as cardiac troponins in determination of AMI (Alpert et al., J. Am. Coll. Cardiol. 36:959-969 (2000)). Troponins have been shown to first increase between 4-12 hours and peak between 10-48 hours after infarction occurs (Goldmann et al., Curr. Control. Trials Cardiovasc. Med. 2:75-84 (2001)).

Until recently, necrosis has been regarded as the sole cause of tissue damage in AMI. However, recent studies indicate that apoptosis also plays an important role in the process of myocardial tissue damage in human AMI (Saraste et al., Circulation 95:320-323 (1997)).

Although both necrosis and apoptosis result in the death of the cell, they differ in several morphological and cellular regulatory features. Necrosis of the heart tissue following AMI is characterized by the rapid loss of cellular homeostasis, rapid swelling as a result of influx of water and extra cellular ions (electrolytes), early plasma membrane rupture, and the disruption of cellular organelles. Due to the membrane rupture and subsequent leakage of a broad array of cellular materials, necrosis induces an inflammatory response and leukocytosis of the heart muscle.

Unlike necrosis, apoptosis associated with AMI is a regulated and energy requiring process that is characterized by shrinkage of the cell and the condensed nuclear chromatin. The cell subsequently detaches from the surrounding tissue by budding out from its membrane to form apoptotic bodies which are rapidly phagocytosed or degraded. Apoptosis of cardiomyocytes is known to be triggered by reperfusion injury and has been shown to occur as early as 3 hours and persist up to 22 hours following the initial event of AMI (Hofstra et al., Lancet 356: 209-212 (2000)). Unlike necrosis, apoptosis of cardiomyocytes does not generally trigger an inflammatory response.

Approximately 1 million patients in the U.S. visit the hospital each year as the result of an AMI. Another 200,000 to 300,000 individuals who suffer an AMI die before medical help is sought. One third of patients who experience ST-elevation die within 24 hours of the onset of sudden ischemia, and many of the survivors experience significant morbidity.

During emergency treatment of AMI, cardiopulmonary resuscitation is administered before the patient is admitted to an intensive cardiac care unit and placed on a cardiac monitor. Oxygen, cardio-tonic drugs, anti-arrhythmic agents, and anticoagulants can be administered as well. However, there has been no FDA approved agent that directly intervenes and thereby reduces cellular damage to the cardiac tissues in victims of AMI. Therefore, there is a need for the development of new therapeutic agents that reduce cellular damage to the myocardium resulting from myocardial infarction.

### SUMMARY OF THE INVENTION

TP508, a polypeptide which stimulates or activates a non-proteolytically activated thrombin receptor (hereinafter “NPAR”), can be used to treat acute myocardial infarction. TP508 can limit damage to myocardial tissues that occurs with acute myocardial infarction. The present invention includes methods of treating myocardial tissue in a subject (e.g., a human patient) having an acute myocardial infarction, comprising administering to the subject a therapeutically effective amount of an NPAR agonist.

In the methods of the invention, the NPAR agonist is a thrombin peptide derivative disclosed herein. More specifically, one thrombin peptide derivative comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. In another specific embodiment, the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative comprising at least eighteen amino acid residues. X₁ is Glu or Gln and X₂ is Phe, Met, Leu, His or Val. In another specific embodiment, the thrombin peptide derivative is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

In another embodiment, the NPAR agonist is a modified thrombin peptide derivative disclosed herein. In a specific embodiment, the modified thrombin peptide derivative comprises the polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acid residues. In another specific embodiment, the modified thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5.

The pharmaceutical compositions comprising thrombin peptide derivatives or modified thrombin peptide derivatives of the present invention can also include a dimerization inhibitor. A dimerization inhibitor is a compound that inhibits or reduces dimerization of a thrombin peptide derivative or a modified thrombin peptide derivative. Dimerization inhibitors include chelating agents and/or thiol-containing compounds.

In another embodiment, the NPAR agonist is a dimer of two thrombin peptide derivatives disclosed herein. More specifically, one such dimer comprises the amino acid sequence Arg-Gly-Asp-Ala-Cys-X₁-Gly-Asp-Ser-Gly-Gly-Pro-X₂-Val (SEQ ID NO:1) or a C-terminal truncated fragment thereof having at least six amino acid residues. In another specific embodiment, the dimer comprises a polypeptide having the amino acid sequence Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), or a fragment thereof comprising amino acid residues 10-18 of SEQ ID NO:2. In another embodiment of the invention, the dimer comprises the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3). In another specific embodiment, the dimer is represented by the structural formula (IV).

The thrombin referred to above can be a mammalian thrombin, and in particular, a human thrombin. The portion of thrombin can be a thrombin receptor binding domain or a portion thereof. In one embodiment, the thrombin receptor binding domain or portion thereof comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the polypeptide Glu-Gly-Lys-Arg-Gly-Asp-Ala-Cys-Glu-Gly (SEQ ID NO:7).

### BRIEF DESCRIPTION OF THE DRAWINGS

FIGS. 1A and 1B are graphs showing coronary microvascular responses to endothelial-dependent adenosine diphosphate (ADP) (FIG. 1A) and endothelial-independent sodium nitroprusside (SNP) (FIG. 1B) substances in TP508 (“TP,” n=7) treated and vehicle control (“CT,” n=7) treated groups of male Yucatan pigs. The graph shows relaxation responses defined as the percent relaxation of the pre-contracted diameter.

FIG. 2 is a graph showing the infarct sizes as a percentage of the area-at-risk (AAR). OVC: normal-cholesterolemic non-treated (n=7); OTC: normal-cholesterolemic treated (n=7); OVH: hyper-cholesterolemic non-treated (n=7); OTH: hyper-cholesterolemic treated (n=7); and OTHF: hyper-cholesterolemic double dose (n=4).

FIG. 3 is a graph showing the area-at-risk (AAR) as a percentage of the total left ventricular mass. OVC: normal-cholesterolemic non-treated (n=7); OTC: normal-cholesterolemic treated (n=7); OVH: hyper-cholesterolemic non-treated (n=7); OTH: hyper-cholesterolemic treated (n=7); and OTHF: hyper-cholesterolemic double dose (n=4).

FIG. 4 is a graph showing the infarct size of normal-cholesterolemic (NC) pigs treated with TP508 (NC-TP508; bolus: 0.5 mg/kg; infusion: 1.25 mg/kg/hr; n=7) as a percentage of the infarct size of the normal-cholesterolemic non-treated vehicle control group (NC-control; saline; n=7). The p value was less than 0.05 (p<0.05).

FIG. 5 is a graph showing the infarct size of hyper-cholesterolemic (HC) pigs treated with TP508 as a percentage of the infarct size of the hyper-cholesterolemic non-treated vehicle control group (A; HC-control; n=7). The treatment groups received either: (1) an intravenous bolus dose of 0.05 mg/kg and an infusion dose of 0.125 mg/kg/hr of TP508 (B; HC-TP508 Dose 0.1×; n=7); (2) an intravenous bolus dose of 0.5 mg/kg and an infusion dose of 1.25 mg/kg/hr of TP508 (C; HC-TP508 Dose 1×; n=7); (3) an intravenous bolus dose of 1.0 mg/kg and an infusion dose of 2.50 mg/kg/hr of TP508 (D; HC-TP508 Dose 2×; n=7); or (4) an equivalent dose of TP508 dimer on a molar basis to HC-TP508 Dose 1× (E; HC-TP508 Dimer; n=7). The p value was less than 0.05 (p<0.05).

### DETAILED DESCRIPTION OF THE INVENTION

The invention includes methods of treating acute myocardial infarction in a subject, for example a human patient, comprising administering to the subject a therapeutically effective amount of an agonist of a non-proteolytically activated thrombin receptor (NPAR) within 7 days of the onset of the acute myocardial infarction. In one embodiment, the NPAR agonist reduces or limits myocardial tissue damage by inhibiting or reducing apoptosis of myocardial tissue.

As used herein, “acute myocardial infarction” refers to a sudden or immediate (not chronic) ischemic event characterized by rapid myocardial tissue damage as a result of insufficient arterial blood flow or oxygen supply. Acute myocardial infarction is characterized by elevation of serum concentration of biomarkers including, but not limited to, troponins I and T, and creatine kinase (CK-MB). An elevated serum level of troponins I and T and CK-MB associated with acute myocardial infarction is defined as exceeding the 99^{th} percentile of a reference control group (i.e. normal population). For example, 99^{th} percentile cutoff point for determining acute myocardial infarction ranges between 0.03-1.0 ng/ml for troponins I and T depending on sensitivity and type of commercially available assays (see, Thygesen et al. Circulation 116:2634-2653 (2007); Morrow et al. Clinical Chemistry 53:552-574 (2007)).

As referred to herein, “acute myocardial infarction” occurs from the onset of a sudden or immediate ischemic event and lasts up to 7 days thereafter. Acute myocardial infarction as used herein is further classified as in “evolving stage” up to 6 hours following the onset of a sudden or immediate ischemic event. Acute myocardial infarction is further classified as “acute stage” from 6 hours to 7 days following the onset of a sudden or immediate ischemic event.

The NPAR agonist is administered anytime during the evolving and/or acute stage(s) including in multiple doses administered at a number of intervals. The NPAR agonist can be administered at the time of and/or within 1, 2, 3, 4, 5, or 6 hour(s) after the onset of a sudden or immediate ischemic event. The NPAR agonist can be administered at the time of and/or within 7, 8, 9, 10, 15, 24, 36 or 48 hours after the onset of acute myocardial infarction. The NPAR agonist can be administered at the time of and/or within 3, 4, 5, 6 or 7 days after the onset of acute myocardial infarction. Alternatively, the NPAR agonist can be administered between 0-48 hours, 1-3 days, 2-4 days, 2-7 days, or 3-7 days following the onset of acute myocardial infarction as used herein. A bolus dose can be administered intravenously, or can be administered by continuous infusion over a period of time. Combinations of bolus and continuous infusion methods can also be used. The NPAR agonist can be administered once during these time periods, or, alternatively, two, three, four, five, six, seven, eight, or even more times within these time periods. In one embodiment, the treatment with the NPAR agonist ends after these time periods; alternatively, treatment can continue after the time period ends.

Compounds which stimulate an NPAR are said to be NPAR agonists. One such NPAR is a high-affinity thrombin receptor present on the surface of most cells. This NPAR component is largely responsible for high-affinity binding of thrombin, proteolytically inactivated thrombin, and thrombin derived peptides to cells. This NPAR appears to mediate a number of cellular signals that are initiated by thrombin independent of its proteolytic activity (see Sower, et. al., Experimental Cell Research, 247:422 (1999)). This NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

A thrombin receptor binding domain is defined as a polypeptide or portion of a polypeptide which directly binds to the thrombin receptor and/or competitively inhibits binding between high-affinity thrombin receptors and alpha-thrombin.

NPAR agonists of the present invention include thrombin derivative peptides, modified thrombin derivative peptides and thrombin derivative peptide dimers as disclosed herein.

### Thrombin Peptide Derivatives

Among NPAR agonists are thrombin peptide derivatives (also: “thrombin derivative peptides”), which are analogs of thrombin that have an amino acid sequence derived at least in part from that of thrombin and are active at a non-proteolytically activated thrombin receptor. Thrombin peptide derivatives include, for example, peptides that are produced by recombinant DNA methods, peptide dimers, peptides produced by enzymatic digestion of thrombin, and peptides produced synthetically, which can comprise amino acid substitutions compared to thrombin, and/or modified amino acid residues, especially at the termini.

It is to be understood that all peptides described herein contain ionizable groups (i.e., the amino group of the N-terminal reside, the carboxyl group of the C-terminal residue and/or the amino acids in the side chains of the peptides). A person having ordinary skill in the art would understand that these ionizable groups contribute to the net charge of the thrombin peptide derivatives as referred to herein, in addition to the pH of the solution in which these peptides exist. As ionic substances which can be present in an acid or base form, the thrombin peptide derivatives as referred to herein can exist in various salt forms depending on their ionization state. Therefore, it is to be understood that when a thrombin peptide derivative is described herein by amino acid sequence or by some other description, corresponding pharmaceutically suitable salts thereof are also included.

Pharmaceutically acceptable salt forms include pharmaceutically acceptable acidic/anionic or basic/cationic salts. Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

NPAR agonists of the present invention include thrombin derivative peptides described in U.S. Pat. Nos. 5,352,664 and 5,500,412. In one embodiment, the NPAR agonist of the present invention is a thrombin peptide derivative or a physiologically functional equivalent, i.e., a polypeptide with no more than about fifty amino acid residues, preferably no more than about thirty amino acid residues and having sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR. The thrombin peptide derivatives or modified thrombin peptide derivatives described herein preferably have from about 12 to about 23 amino acid residues, more preferably from about 19 to about 23 amino acid residues.

In another embodiment, the NPAR agonist of the present invention is a thrombin peptide derivative comprising a moiety represented by Structural Formula (I):

Asp-Ala-R (I).

R is a serine esterase conserved domain. Serine esterases (e.g., trypsin, thrombin, chymotrypsin and the like) have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in particulars which do not affect the function of the thrombin receptor binding domain or the serine esterase conserved amino acid sequence. Such particulars may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing 4-12 of the N-terminal amino acid residues of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁ is either Ala or Ser; X₂ is either Glu or Gln; and X₃ is Phe, Met, Leu, His, or Val.

In one embodiment, the serine esterase conserved sequence comprises the amino acid sequence Cys-Glu-Gly-Asp-Ser-Gly-Gly-Pro-Phe-Val (SEQ ID NO:14) or a C-terminal truncated fragment of a polypeptide having the amino acid sequence of SEQ ID NO:14. It is understood, however, that zero, one, two or three amino acid residues in the serine esterase conserved sequence can differ from the corresponding amino acid in SEQ ID NO:14. Preferably, the amino acid residues in the serine esterase conserved sequence which differ from the corresponding amino acid in SEQ ID NO:14 are conservative substitutions, and are more preferably highly conservative substitutions. A “C-terminal truncated fragment” refers to a fragment remaining after removing an amino acid residue or block of amino acid residues from the C-terminus, said fragment having at least six and more preferably at least nine amino acid residues.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acid residues, preferably at least nine amino acid residues.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO:1). X₁ and X₂ are as defined above. The thrombin peptide derivative can comprise the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or an N-terminal truncated fragment thereof, provided that zero, one, two or three amino acid residues at positions 1-9 in the thrombin peptide derivative differ from the amino acid residue at the corresponding position of SEQ ID NO:6. Preferably, the amino acid residues in the thrombin peptide derivative which differ from the corresponding amino acid residues in SEQ ID NO:6 are conservative substitutions, and are more preferably highly conservative substitutions. An “N-terminal truncated fragment” refers to a fragment remaining after removing an amino acid residue or block of amino acid residues from the N-terminus, preferably a block of no more than six amino acid residues, more preferably a block of no more than three amino acid residues.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a substituted or unsubstituted aliphatic group comprising up to 10 carbon atoms, or Rₐ and R_{b}, taken together with the nitrogen to which they are bonded, form a C₃-C₁₀ non-aromatic heterocyclic group, and said N-terminal acyl group is represented by R_{c}C(O)—, wherein R_{c} is hydrogen, a substituted or unsubstituted aliphatic group comprising up to 10 carbon atoms, or a C₃-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivatives comprising the amino acid sequences SEQ ID NO: 6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acid residues at positions 1-9 and 14-23 in the thrombin peptide derivative can differ from the corresponding amino acid in SEQ ID NO:6. It is also understood that zero, one, two or three amino acid residues at positions 1-14 and 19-33 in the thrombin peptide derivative can differ from the corresponding amino acid in SEQ ID NO:18. Preferably, the amino acid residues in the thrombin peptide derivative which differ from the corresponding amino acid in SEQ ID NO:6 or SEQ ID NO:18 are conservative substitutions, and are more preferably highly conservative substitutions. Alternatively, an N-terminal truncated fragment of the thrombin peptide derivative having at least fourteen amino acid residues or a C-terminal truncated fragment of the thrombin peptide derivative having at least eighteen amino acid residues is a thrombin peptide derivative to be used as an NPAR agonist.

A “C-terminal truncated fragment” refers to a fragment remaining after removing an amino acid or block of amino acid residues from the C-terminus. An “N-terminal truncated fragment” refers to a fragment remaining after removing an amino acid residue or block of amino acid residues from the N-terminus. It is to be understood that both C-terminal truncated fragments and N-terminal truncated fragments can optionally be amidated at the C-terminus and/or acylated at the N-terminus.

A preferred thrombin peptide derivative for use in the disclosed methods comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2). Another preferred thrombin peptide derivative for use in the disclosed method comprises the polypeptide Asp-Asn-Met-Phe-Cys-Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe (SEQ ID NO:19). X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acid residues, or C-terminal truncated fragments of these preferred thrombin peptide derivatives, the C-terminal truncated fragments having at least eighteen amino acid residues, can also be used in the disclosed methods.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acid residues, or C-terminal truncated fragments of TP508 (or deamide TP508), the C-terminal truncated fragments having at least eighteen amino acid residues.

As used herein, a “conservative substitution” in a polypeptide is the replacement of an amino acid with another amino acid that has the same net electronic charge and approximately the same size and shape. Amino acid residues with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acid residues with phenyl or substituted phenyl groups in their side chains are considered to have about the same size and shape. Listed below are five groups of amino acids. Replacing an amino acid residue in a polypeptide with another amino acid residue from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

### Modified Thrombin Peptide Derivatives

In one embodiment of the invention, the NPAR agonists are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, and an S-protected cysteine. Preferably, cysteine is replaced with alanine or serine. The modified thrombin peptide derivatives have about the same biological activity as the unmodified thrombin peptide derivatives.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acids. More specifically, the thrombin peptide derivative comprises the polypeptide Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val (SEQ ID NO:20), or a fragment thereof comprising amino acid residues 10-18 of SEQ ID NO:20. Even more specifically, the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5. Xaa is alanine, glycine, serine or an S-protected cysteine. X₁ is Glu or Gln and X₂ is Phe, Met, Leu, His or Val. In one embodiment, X₁ is Glu, X₂ is Phe, and Xaa is Ala. In another embodiment, X₁ is Glu, X₂ is Phe, and Xaa is Ser. One example of a thrombin peptide derivative of this type is the polypeptide Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22), wherein H is a hydrogen atom of alanine indicating no modification at the N-terminus, and NH₂ indicates amidation at the C-terminus as —C(O)NH₂. Zero, one, two or three amino acids in the thrombin peptide derivative differ from the amino acid at the corresponding position of SEQ ID NO:4, 20, 5, 21 or 22, provided that Xaa is alanine, glycine, serine and an S-protected cysteine. Preferably, the difference is conservative.

In another specific embodiment, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe (SEQ ID NO:23), or a fragment thereof comprising amino acids 6-28. More preferably, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe (SEQ ID NO:24), or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X₁ is Glu, X₂ is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe (SEQ ID NO:25). A further example of a thrombin peptide derivative of this type is the polypeptide H-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26), wherein His a hydrogen atom of aspartic acid indicating no modification at the N-terminus, and NH₂ indicates amidation at the C-terminus as —C(O)NH₂. Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g., cysteine-S—S-t-Bu). Preferably the protective group is an alkyl thioether. More preferably, the S-protected cysteine is an S-methyl cysteine. Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

The thrombin peptide derivatives or the modified thrombin peptide derivatives of the present invention can be mixed with a dimerization inhibitor for the preparation of a pharmaceutical composition comprising the thrombin peptide derivatives or the modified thrombin peptide derivatives of the present invention. Dimerization inhibitors can include chelating agents and/or thiol-containing compounds. An antioxidant can also be used in combination with the chelating agent and/or the thiol-containing compound.

A “chelating agent,” as used herein, is a compound having multiple sites (two, three, four or more) which can simultaneously bind to a metal ion or metal ions such as, for example, lead, cobalt, iron or copper ions. The binding sites typically comprise oxygen, nitrogen, sulfur or phosphorus. For example, salts of EDTA (ethylenediaminetetraacetic acid) can form at least four to six bonds with a metal ion or metal ions via the oxygen atoms of four acetic acid moieties (—CH₂C(O)O⁻) and the nitrogen atoms of ethylenediamine moieties (>N—CH₂—CH₂—N<) of EDTA. It is understood that a chelating agent also includes a polymer which has multiple binding sites to a metal or metal ions. Preferably, a chelating agent of the invention is non-toxic and does not cause unacceptable side effects at the dosages of pharmaceutical composition being administered in the methods of the invention. As a chelating agent of the invention, a copper-chelating agent is preferable. A “copper-chelating agent” refers to a chelating agent which can bind to a copper ion or copper ions. Examples of the copper-chelating agent include ethylenediaminetetraacetic acid (EDTA), penicillamine, trientine, N,N′-diethyldithiocarbamate (DDC), 2,3,2′-tetraamine(2,3,2′-tet), neocuproine, N,N,N′,N′-tetrakis(2-pyridylmethyl)ethylenediamine (TPEN), 1,10-phenanthroline (PHE), tetraethylenepentamine (TEPA), triethylenetetraamine and tris(2-carboxyethyl)phosphine (TCEP). Additional chelating agents are diethylenetriaminepentacetic acid (DTPA) and bathophenanthroline disulfonic acid (BPADA). EDTA is a preferred chelating agent. Typical amounts of a chelating agent present in the pharmaceutical compositions of the instant invention are in a range of between about 0.00001% and about 0.1% by weight, preferably between about 0.0001% and about 0.05% by weight.

A “pharmaceutically acceptable thiol-containing compound” as used herein is a compound which comprises at least one thiol (—SH) group and which does not cause unacceptable side effects at the dosages which are being administered. Examples of a pharmaceutically acceptable thiol-containing compound include thioglycerol, mercaptoethanol, thioglycol, thiodiglycol, cysteine, thioglucose, dithiothreitol (DTT) and dithio-bis-maleimidoethane (DTME). Typically, between about 0.001% and about 5% by weight, preferably between about 0.05% and about 1.0% by weight of a pharmaceutically acceptable thiol-containing compound is present in the pharmaceutical compositions of the invention.

An “antioxidant,” as used herein, is a compound which is used to prevent or reduce an oxidation reaction caused by an oxidizing agent such as oxygen. Examples of antioxidants include tocopherol, cystine, methionine, glutathione, tocotrienol, dimethyl glycine, betaine, butylated hydroxyanisole, butylated hydroxytoluene, vitamin E, ascorbic acid, ascorbyl palmitate, thioglycolic acid and antioxidant peptides such as, for example, turmerin. Typically, between about 0.001% and about 10% by weight, preferably between about 0.01% and about 5%, more preferably between about 0.05% and about 2.0% by weight of an antioxidant is present in the pharmaceutical compositions of the invention.

It is understood that certain chelating agents or thiol-containing compounds may also function as antioxidants, for example, tris(2-carboxyethyl)phosphine, cysteine or dithiothreitol. Other types of commonly used antioxidants, however, do not contain a thiol group. It is also understood that certain thiol-containing compounds may also function as a chelating agent, for example, dithiothreitol. Other types of commonly used chelating agents, however, do not contain a thiol group. It is also understood that the pharmaceutical compositions of the instant invention can comprise more than one chelating agent, thiol-containing compound or antioxidant. That is, for example, a chelating agent can be used either alone or in combination with one or more other suitable chelating agents.

### Thrombin Peptide Derivative Dimers

In some aspects of the present invention, the NPAR agonists of the methods are thrombin peptide derivative dimers. The dimers essentially do not revert to monomers and still have about the same biological activity as the thrombin peptide derivative monomers described above. A “thrombin peptide derivative dimer” is a molecule comprising two thrombin peptide derivatives (polypeptides) linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprise the thrombin peptide derivatives described above. Specifically, thrombin peptide derivatives have fewer than about fifty amino acids, preferably about thirty-three or fewer amino acids. The thrombin peptide derivative dimers described herein are formed from polypeptides typically having at least six amino acids and preferably from about 12 to about 33 amino acid residues, and more preferably from about 12 to about 23 amino acid residues. Thrombin peptide derivative monomer subunits of the dimers have sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6)) so that NPAR is activated.

In a specific embodiment, each of the two thrombin peptide derivatives (monomers) of a dimer comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. More specifically, a polypeptide monomer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or a fragment thereof comprising amino acid residues 10-18 of SEQ ID NO:5. Even more specifically, a polypeptide monomer comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), or a fragment thereof comprising amino acid residues 10-18 of SEQ ID NO:2. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3), wherein H signifies a hydrogen atom of alanine indicating no modification at the N-terminus, and NH₂ signifies amidation at the C-terminus as —C(O)NH₂. Zero, one, two or three amino acid residues in the polypeptide differ from the amino acid residue at the corresponding position of SEQ ID NO:6, 1, 2, or 3. Preferably, the difference is conservative.

One example of a thrombin peptide derivative dimer of the present invention is represented by Formula (IV):

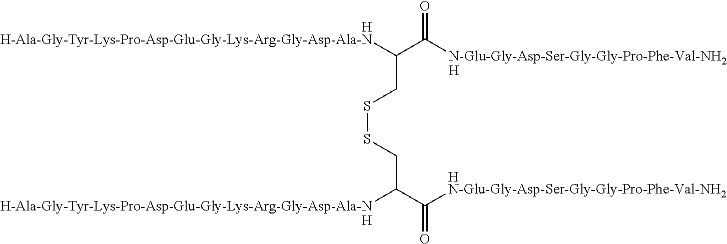

(IV). The dimer of Formula (IV) and salts thereof are referred to as “TP508 dimer.”

In another specific embodiment, each of the two thrombin peptide derivatives (monomers) of a dimer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27), or a C-terminal truncated fragment thereof having at least twenty-three amino acid residues. More preferably, a polypeptide comprises Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr (SEQ ID NO:8), or a C-terminal truncated fragment thereof comprising at least twenty-three amino acid residues. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27). A further example of a polypeptide of this type is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:9), wherein H signifies a hydrogen atom of alanine indicating no modification at the N-terminus, and NH₂ indicates amidation at the C-terminus —C(O)NH₂. Zero, one, two or three amino acid residues in the polypeptide differ from the amino acid residue at the corresponding position of SEQ ID NO:27, 28 or 29. Preferably, the difference is conservative.

Methods of Treatment with NPAR Agonists

The present invention is directed to methods of treating acute myocardial infarction in a subject, comprising administering to the subject a therapeutically effective amount of an NPAR agonist within 7 days following the onset of acute myocardial ischemia.

The disclosed methods can be used as a primary treatment method during the occurrence of acute myocardial infarction. The disclosed methods are not limited to any particular kind of cardiac tissue or location of the heart. Examples of an acute myocardial infarction which can be treated by the disclosed methods include, but, are not limited to, an infarction to the myocardium of septal wall, anterior wall, anteroseptal wall, anterolateral wall, extensive anterior wall, inferior wall, lateral wall posterior wall, left ventricular wall and right ventricular wall. One example of an acute myocardial infarction which can be treated by the disclosed methods include, but, are not limited to, an infarction to the endocardium.

A “subject” is preferably a human, but can also be an animal in need of treatment with a thrombin receptor agonist, e.g., companion animals (e.g., dogs, cats, and the like), farm animals (e.g., cows, pigs, horses and the like) and laboratory animals (e.g., rats, mice, guinea pigs and the like). As used herein, a “subject” can be a human patient undergoing acute myocardial infarction. In one embodiment, a “subject” is a hypercholesterolemic patient whose cholesterol level is above 200 mg per decilitre (mg/dL). In another embodiment, a hypercholestrolemic patient has a total cholesterol level that is 240 mg/dL or higher. As used herein, a “subject” can be a hypercholesterolemic patient whose low-density lipoproteins (LDL) level is 70 mg/dL or higher. In another embodiment, a subject can be a human patient undergoing acute myocardial infarction, whose LDL level is 100, 130, 160, 190 mg/dL or higher. In yet another embodiment, a “subject” can be a human patient whose triglyceride level is 200 mg/dL or higher.

“Hypercholesterolemia” or “hypercholesterolemic” as used herein refers to a condition that a subject's total cholesterol levels in the blood have been above 200 mg/dL for 1, 2, 3, 4, 5, 6, 7 day(s) or longer immediately prior to the onset of acute myocardial infarction. In one embodiment, “hypercholesterolemia” or “hypercholesterolemic” as used herein refers to a condition that a subject's total cholesterol levels in the blood have been above 200 mg/dL for at least 1, 2, 3, or 4 week(s) immediately prior to the onset of acute myocardial infarction. In another embodiment, “hypercholesterolemia” or “hypercholesterolemic” refers to a condition that a subject's total cholesterol levels have been above 200 mg/dL for at least 1, 2, 3, 4, 6 month(s) or longer prior to the onset of acute myocardial infarction.

A “therapeutically effective amount” is the quantity of the NPAR agonist that results in a decreased amount of damage to the myocardium compared to untreated or sham-treated controls. NPAR agonists can effectively inhibit or reduce the extent of apoptosis. A therapeutically effective amount can be a quantity of NPAR agonist that results in a reduced extent of apoptosis in the myocardium, as compared to untreated or sham-treated controls. This can be determined by: (1) smaller infarct size; (2) lower protein factors associated with apoptosis (Apoptosis Inducing Factors (AIF), bad, and cleaved-caspase 3, etc.), in the myocardium; and/or (3) fewer TUNEL-positive cardiomyocytes as compared to an untreated population. The amount of the NPAR agonist administered will depend on the degree of severity of acute myocardial infarction, and the release characteristics of the pharmaceutical formulation. It will also depend on the subject's health, size, weight, age, sex and tolerance to drugs. When administered more than once, the NPAR agonists are preferably administered at evenly spaced intervals; each dose can be the same or different, but is preferably the same. A dose delivered to the ischemic site can be, for example, 0.1-500 μg, preferably 1-50 μg of NPAR agonist, and is commonly 3, 5, 10, 30 or 50 μg.

The disclosed NPAR agonists can be administered by any suitable route, including, for example, by local introduction to the ischemic site by, for example, cardiac catheterization. The NPAR agonist can be administered intravenously. The NPAR agonist can be administered to the subject in a sustained release formulation, or can be delivered by a pump or an implantable device, or by an implantable carrier such as the polymers discussed below. “Administered to the cardiac tissue” means delivered to the inner or outer surfaces of the heart. Alternatively, the point of delivery of the NPAR agonist can be in sufficient proximity to the ischemic site or surfaces of the heart so that the agonist can diffuse and contact the ischemic site or heart surfaces, for example, within 1 cm of one or both ischemic site or surfaces of the heart.

The NPAR agonists can be administered to the subject in conjunction with an acceptable pharmaceutical carrier as part of a pharmaceutical composition. The formulation of the pharmaceutical composition will vary according to the mode of administration selected. Suitable pharmaceutical carriers may contain inert ingredients which do not interact with the NPAR agonist. The carriers should be biocompatible, i.e., non-toxic, non-inflammatory, non-immunogenic and devoid of other undesired reactions at the administration site. Examples of pharmaceutically acceptable carriers include, for example, saline, commercially available inert gels, or liquids supplemented with albumin, methyl cellulose or a collagen matrix. Further examples include sterile water, physiological saline, bacteriostatic saline (saline containing about 0.9% mg/ml benzyl alcohol), phosphate-buffered saline, Hank's solution, Ringer's-lactate and the like. Standard pharmaceutical formulation techniques can be employed, such as those described in Remington's Pharmaceutical Sciences (Remington's Pharmaceutical Sciences. XVIII, Mack Publishing Company, Easton, Pa. (1990)).

Pharmaceutical compositions may include gels. Gels are compositions comprising a base selected from an oleaginous base, water, or an emulsion-suspension base. To the base is added a gelling agent which forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent or can be mixed after the gelation process.

In one embodiment, the NPAR agonists are administered in a sustained release formulation. Polymers are often used to form sustained release formulations. Examples of these polymers include poly α-hydroxy esters such as polylactic acid/polyglycolic acid homopolymers and copolymers, polyphosphazenes (PPHOS), polyanhydrides and poly (propylene fumarates).

Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson, et al., Adv. Drug Deliv. Rev. 28:5 (1997), the entire teachings of which are incorporated herein by reference). The incorporation of poly-ethylene glycol into the polymer as a blend to form microparticle carriers allows further alteration of the release profile of the active ingredient (see Cleek et al., J. Control Release 48:259 (1997), the entire teachings of which are incorporated herein by reference). Ceramics such as calcium phosphate and hydroxyapatite can also be incorporated into the formulation to improve mechanical qualities.

PPHOS polymers contain alternating nitrogen and phosphorous with no carbon in the polymer backbone, as shown below in Structural Formula (II):

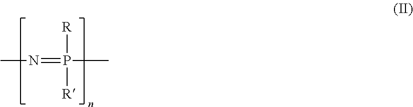

The properties of the polymer can be adjusted by suitable variation of side groups R and R′ that are bonded to the polymer backbone. For example, the degradation of and drug release by PPHOS can be controlled by varying the amount of hydrolytically unstable side groups. With greater incorporation of either imidazolyl or ethylglycol substituted PPHOS, for example, an increase in degradation rate is observed (see Laurencin et al., J Biomed Mater. Res. 27:963 (1993), the entire teachings of which are incorporated herein by reference), thereby increasing the rate of drug release.

Acute myocardial infarction is often accompanied by symptoms and infirmities such as chest pain and inflammation due to the necrotic processes in the damaged cardiac tissue. In certain instances it may be advantageous to co-administer one or more additional pharmacologically active agents along with an NPAR agonist to address such issues. For example, managing pain and inflammation may require co-administration with analgesic and/or anti-inflammatory agents. Thrombolytic agents such as tissue plasminogen activator (tPA), blood thinning agents such as heparin, anti-arrhythmic agents such as sodium, calcium or potassium channel blockers (quinidine, lidocaine, propafenone, bretylium, verapamil, etc.) as well as beta blockers (propranolol and sotalol) can be also co-administered.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these six articles are incorporated herein by reference in their entirety.

Thrombin peptide derivative dimers can be prepared by oxidation of the monomer. Thrombin peptide derivative dimers can be prepared by reacting the thrombin peptide derivative with an excess of oxidizing agent. A well-known suitable oxidizing agent is iodine.

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

The term “aryl group” includes both carbocyclic and heterocyclic aromatic ring systems. Examples of aryl groups include phenyl, indolyl, furanyl and imidazolyl.

An “aliphatic group” is a straight chain, branched or cyclic non-aromatic hydrocarbon. An aliphatic group can be completely saturated or contain one or more units of unsaturation (e.g., double and/or triple bonds), but is preferably saturated, i.e., an alkyl group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. Aliphatic groups include, for example, methyl, ethyl, n-propyl, iso-propyl, n-butyl, sec-butyl, tert-butyl, pentyl, cyclopentyl, hexyl, cyclohexyl, octyl and cyclooctyl.

Suitable substituents for an aliphatic group, an aryl group or a non-aromatic heterocyclic group are those which do not significantly lower therapeutic activity of the NPAR agonist, for example, those found on naturally occurring amino acids. Examples include —OH, a halogen (—Br, —Cl, —I and —F), —O(Rₑ), —O—CO—(Rₑ), —CN, —NO₂, —COOH, ═O, —NH₂—NH(Rₑ), —N(Rₑ)₂. —COO(Rₑ), —CONH₂, —CONH(Rₑ), —CON(Rₑ)₂, —SH, —S(Rₑ), an aliphatic group, an aryl group and a non-aromatic heterocyclic group. Each Rₑ is independently an alkyl group or an aryl group. A substituted aliphatic group can have more than one substituent.

The invention is illustrated by the following example which is not intended to be limiting in any way.

### Example 1

### I. Experimental Procedures

Hypercholesterolemic Yucatan miniswine were divided randomly into three treatment groups. These groups were: (1) placebo (n=5-10) (2) low dose TP508 treatment (n=6-10) and (3) high dose TP508 treatment (n=4). All animals were subjected to regional left ventricular (LV) ischemia by left anterior descending (LAD) arterial occlusion distal to the second diagonal branch for 60 minutes. The treatment groups received an intravenous (IV) bolus dose of either placebo or TP508 10 minutes prior to the onset of reperfusion, followed by a constant IV infusion of TP508 or vehicle for the remainder of the experiment. The myocardium was reperfused for 120 minutes following ischemia. Arterial blood gas (ABG), arterial blood pressure, hematocrit (Hct), LV pressure, heart rate (HR), EKG/ECG, O₂ saturation, core temperature, and intravenous fluid requirements were measured and recorded. Myocardial segmental shortening in the long axis (parallel to the LAD) and short-axis (perpendicular to the LAD) was recorded at baseline prior to the onset of ischemia, and prior to harvest after 120 minutes of reperfusion. Upon completion of the protocol, the heart was excised, and tissue samples from the ischemic-reperfused, and distal LAD territory were collected for molecular analyses as described below. Additionally, an appropriate amount of a blood sample was collected and frozen for possible future analysis of additional biomarkers.

### Surgery

Swine were sedated with ketamine hydrochloride (20 mg/kg, intramuscularly, Abbott Laboratories, North Chicago, Ill.), and anesthetized with a bolus infusion of thiopental sodium (Baxter Healthcare Corporation, Inc, Deerfield, Ill.; 5.0 to 7.0 mg/kg intravenously), followed by endotracheal intubation. Ventilation began with a volume-cycled ventilator (model Narkomed II-A; North American Drager, Telford, Pa.; oxygen, 40%; tidal volume, 600 cc; ventilation rate, 12 breaths/min; positive end-expiratory pressure, 3 cm H₂O; inspiratory to expiratory time, 1:2). General endotracheal anesthesia was established with 3.0% sevoflurane (Ultane; Abbott Laboratories) at the beginning of the surgical preparation, and maintained with 1.0% throughout the experiment. One liter of Lactated Ringer's intravenous (IV) fluid was administered after induction of anesthesia and continued thereafter throughout the surgical protocol at 150 cc/hour. A right groin dissection was performed and the femoral vein and common femoral artery were isolated and cannulated utilizing 8F sheaths (Cordis Corporation, Miami, Fla.). The femoral vein was cannulated for intravenous access, TP508/placebo delivery, and the right common femoral artery was cannulated for arterial blood sampling and continuous intra-arterial blood pressure monitoring (Millar Instruments, Houston, Tex.). A median sternotomy was performed exposing the pericardial sac, which was then opened to form a pericardial cradle. A catheter-tipped manometer (Millar Instruments, Houston, Tex.) was introduced through the apex of the left ventricle to record LV pressure. Segmental shortening in the area-at-risk (AAR) was assessed utilizing a sonometric digital ultrasonic crystal measurement system (Sonometrics Corp, London, ON, Canada) using four 2-mm digital ultrasonic probes implanted in the subepicardial layer approximately 10 mm apart within the ischemic LV area. Cardiosoft software (Sonometrics Corp, London, ON, Canada) was used for data recording (LV dP/dt, segmental shortening, arterial blood pressure, heart rate) and subsequent data analysis to determine myocardial function. Baseline hemodynamic, functional measurement (global: +LV dP/dt, regional: segmental shortening), arterial blood gas analysis, and hematocrit were obtained. ABG analysis was continued every 15 minutes throughout the protocol and hematocrit was measured every 20 minutes. All animals received 75 mg of lidocaine and 20 mEq of potassium chloride as prophylaxis against ventricular dysrhythmia, as well as 60 units/kg of intravenous heparin bolus prior to occlusion of the LAD. The LAD coronary artery was occluded 3 mm distal to the origin of the second diagonal branch utilizing a Rommel tourniquet. Myocardial ischemia was confirmed visually by regional cyanosis of the myocardial surface. Fifty minutes after the initiation of regional ischemia (10 minutes prior to the onset of reperfusion), control pigs received a placebo carrier solution infusion intravenously, and treatment animals received TP508. The Rommel tourniquet was released 60 minutes after the onset of acute ischemia and the myocardium was reperfused for 120 minutes. At the end of the reperfusion period, hemodynamic and functional measurements were recorded as described above, followed by re-ligation of the LAD and injection of monastryl blue pigment (Engelhard Corp, Louisville, Ky.) at a 1:150 dilution in PBS into the aortic root after placement of an aortic crossclamp distal to the coronary arterial ostia to demarcate the area-at-risk (AAR). The heart was rapidly excised and the entire left ventricle, including the septum, was dissected free. The LV was cut in to 1 cm thick slices perpendicular to the axis of the LAD. The AAR was clearly identified by lack of blue pigment staining. Tissue from the AAR of the slice 1 cm proximal to the LV apex was isolated and divided for use in molecular and microvascular studies. The remaining slices were weighed and utilized for infarct size calculation as described below. Ventricular dysrhythmia (ventricular fibrillation or pulseless ventricular tachycardia) events were recorded and treated with immediate electrical cardioversion (50 J, internal paddles).

### Measurement of Global and Regional Myocardial Function

Global myocardial function was assessed by calculating the maximum positive first derivative of LV pressure over time (+dP/dt). Regional myocardial function was determined by using subepicardial 2-mm ultrasonic probes to calculate the percentage segment shortening (% SS), which was normalized to the baseline. Measurements were taken at baseline prior to the onset of ischemia and at the end of reperfusion. The ventilator was stopped during data acquisition to eliminate the effects of respiration. Measurements were made during at least three cardiac cycles in normal sinus rhythm and then averaged. Digital data were inspected for the correct identification of end-diastole and end-systole. End-diastolic segment length (EDL) was measured at the onset of the positive dP/dt, and the end-systolic segment length (ESL) at the peak negative dP/dt.

### Coronary Microvessel Studies

Coronary microvessel studies were performed to examine the effects of TP508 on endothelial and vascular smooth muscle injury after ischemia-reperfusion in the coronary microcirculation. After cardiac harvest, myocardial specimens from the ischemic LAD territory were immersed in 4° C. Krebs solution and coronary arterioles (80 to 130 μM in diameter and 1 to 2 mm in length) were dissected sharply from the surrounding tissue with a 40× magnification-dissecting microscope. Microvessels were mounted and examined in a pressurized isolated organ chamber, as described previously (1). The responses to sodium nitroprusside (SNP) (1 nM to 100 mM), an endothelium-independent cGMP-mediated vasodilator, as well as Substance P (0.1 μM to 10 nM), an endothelium-dependent receptor-mediated vasodilator that acts via bioavailable nitric oxide, were studied after pre-contraction to 20-50% of the baseline diameter with the thromboxane A2 analog U46619 (0.1-1 μM). In addition, coronary microvascular responses to adenosine diphosphate (ADP), an endothelium-dependent vasodilator, was also examined. Relaxation responses are defined as the percent relaxation of the pre-contracted diameter.

### Quantification of Myocardial Infarct Size

The left ventricle was isolated and cut into 1 cm slices and infarct size was assessed. Briefly, slices were immediately immersed in 1% triphenyl tetrazolium chloride (TTC, Sigma Chemical Co, St Louis, Mo.) in phosphate buffer (pH 7.4) at 38° C. for 30 minutes. The infarct area (characterized by absence of staining), the non-infarcted area-at-risk (characterized by red tissue staining), and the non-ischemic portion of the LV (characterized by purple tissue staining) were sharply dissected from one another and weighed. The percentage area-at-risk was defined as: (Infarct mass+non-infarct area-at-risk mass)/Total LV mass×100. Infarct size was calculated as a percentage of area at risk (AAR) to normalize for any variation in AAR size using the following equation: (Infarct mass/total mass AAR)×100.

### Western Blotting for Anti-Apoptosis Factors

Whole-cell lysates were isolated from the homogenized myocardial samples with a radio immunoprecipitation assay (RIPA) buffer (Boston Bioproducts, Worcester, Mass.) and centrifuged at 12,000 g for 10 min at 4° C. to separate soluble from insoluble fractions. Protein concentration was measured spectrophotometrically at a 595-nm wavelength with a DC protein assay kit (Bio-RAD, Hercules, Calif.). Forty to eighty micrograms of total protein were fractionated by 4-20% gradient, SDS polyacrylamide gel electrophoresis (Invitrogen, San Diego, Calif.) and transferred to polyvinylidene difluoride (PVDF) membranes (Millipore, Bedford, Mass.). Each membrane was incubated with specific antibodies (Cell Signaling Technology, Beverly, Mass.) as follows: anti-Apoptosis Inducing Factor (AIF) (1:1000 dilution), anti-Bcl-2 (1:1000 dilution), anti-Bad (1:1000 dilution), anti-phospho Bad (Serine 136) (1:500 dilution), anti-phospho Bad (Serine 112) (1:2000 dilution), anti-caspase-3 (1:1000 dilution), anti-cleaved caspase-3 (1:1000 dilution), anti-TARP (1:1000 dilution), anti-cleaved PARP (1:1000 dilution) and anti-BNIP3. The membranes were subsequently incubated for 1 hour in diluted appropriate secondary antibody (Jackson Immunolab, West Grove, Pa.). Immune complexes were visualized with the enhanced chemiluminescence detection system (Amersham, Piscataway, N.J.). Bands were quantified by densitometry of radioautograph films. Ponceau S staining was performed to confirm equivalent protein loading.

### Serum Creatine Kinase-MB, Troponin I, and Fatty Acid Binding Protein Quantification

Serum collected prior to sacrifice was utilized for quantification of Creatine Kinase-MB (CK-MB), Troponin I, and Fatty Acid Binding Protein (FABP) utilizing a protein microarray (Allied Biotech Inc., Ijamsville, Md.). Serum levels of markers were calculated based on standards provided by the manufacturer.

### Tissue Inflammatory Marker Quantification

Myocardial tissue (˜50 mg) from the area at risk (AAR) was homogenized in RIPA buffer (Boston BioProducts, Worcester, Mass.) with protease inhibitor added (Complete Tablets, Roche Applied Sciences, Indianapolis, Ind.) and centrifuged at 12,000 g for 10 minutes. Supernatants were aliquoted and a cytokine array was utilized (Allied Biotech Inc., Ijamsville, Md.) for detection of interleukin (IL)-6, IL-8, and TNF-α in triplicate. Tissue levels of inflammatory mediators were calculated based on standards provided by the manufacturer.

### Immunohistochemical Staining

Myocardial tissue from the ischemic territory was placed in 10% buffered formalin for 24 hours, followed by paraffin mounting and sectioning into 4 μm slices.

### 1. Poly (ADP) Ribosylation Staining

For the immunohistochemical detection of poly(ADP-ribose) polymerase activity, mouse monoclonal anti-poly(ADP-ribose) (PAR) antibody (Calbiochem, San Diego, Calif.) (1:1000, overnight, 4° C.) was used. Secondary labeling was achieved by using biotinylated horse anti-mouse antibody (Vector Laboratories, Burlingame, Calif.) (30 min room temperature). Horseradish peroxidase-conjugated avidin (30 min, room temperature) and brown colored diaminobenzidine (˜6 min, room temperature) were used to visualize the labeling (Vector Laboratories, Burlingame, Calif.). The sections were counterstained with hematoxylin (blue color). The intensity of specific staining of individual sections was determined by a blinded experimenter. The semiquantitative PAR-positivity score was the following: 1: no specific staining, 2: light cytoplasmic staining, 3: few positive nuclei, 4: light nuclear staining in approximately 10% of cells, 5: light nuclear staining in approximately 25% of cells, 6: light nuclear staining in approximately 50% of cells, 7: strong nuclear staining in approximately 50% of cells, 8: approximately 75% of the nuclei are positive, 9: approximately 90% of the nuclei were positive, 10: few negative cells.

### 2. TUNEL Staining

The apoptotic cells were identified by dUTP nick-end labeling (TUNEL) using an apoptosis detection kit according to the manufacturer's protocol (Chemicon Inc, Temecula, Calif.). Five photographs (magnification 20×) of each tissue section were taken. The nuclei were viewed and manually counted by an observer blinded to the experimental conditions. The number of TUNEL-positive cardiomyocytes, indicating apoptosis, was expressed in mean number per/100 cells/microscopic field.

### Statistical Methods

Data was reported as means±SEM. Microvessel responses were expressed as percent relaxation of the preconstricted diameter and analyzed using two-way, repeated measures analysis of variance examining the relationship between vessel relaxation, log concentration of the vasoactive agent of interest, and the experimental group (SAS Version 9.1, Cary, N.C.). Western blots were expressed as a ratio of protein to loading band density and analyzed after digitization and quantification of X-ray films with Image J version 1.33 (National Institutes of Health, USA). Blots and ILM data were analyzed using analyses of variance. Bonferroni corrections were applied to multiple tests and probability values of less than 0.05 were considered statistically significant.

### II. Results

1. Fourteen male Yucatan pigs underwent 60 min of mid-left anterior descending artery occlusion followed by 120 min of reperfusion according to the procedure described in Section I above. Among the fourteen male Yucatan pigs, seven pigs received vehicle control solution (CT, n=7) and the other seven pigs received TP508 (TP, n=7). Myocardial function was monitored throughout the experiments as described above. Coronary microvascular responses were examined to endothelial-dependent (ADP) and endothelial-independent (SNP) substances. Monastryl blue/TTC staining was utilized to measure the area-at-risk (AAR) and necrosis. Expression of apoptotic related proteins was examined by Western Blotting. TUNEL staining was utilized to quantify the magnitude of apoptosis in the ischemic and non-ischemic areas. The bolus dose was 500 μg/kg, followed by a continuous IV infusion of 1.25 mg/kg/hr.

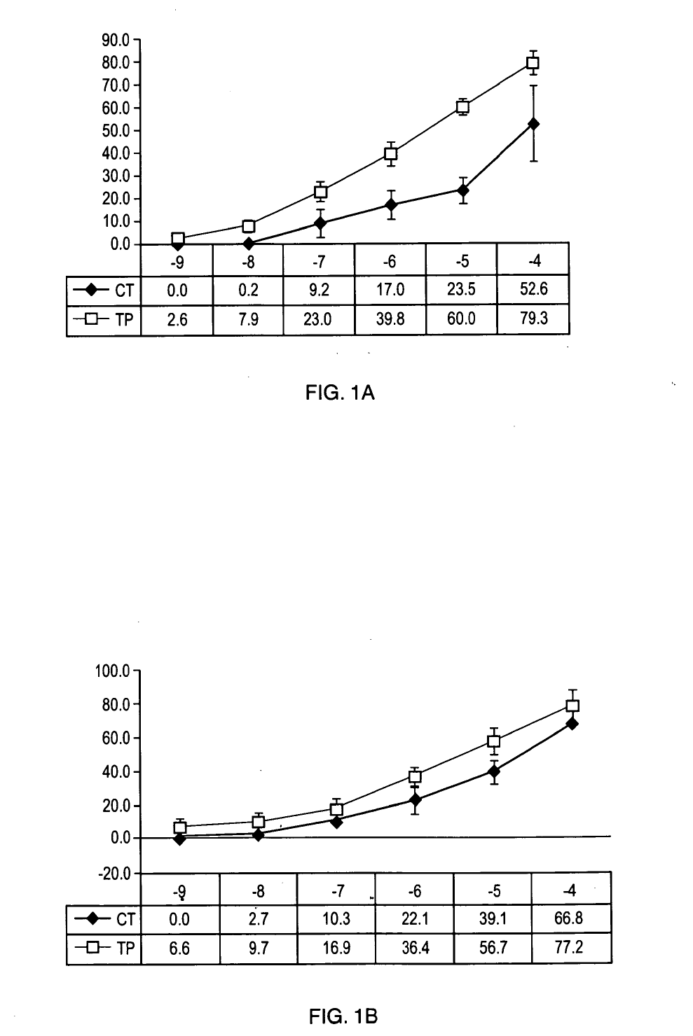

Cardiac function was not significantly different between groups. The coronary microvascular responses to endothelial-dependent ADP and the endothelial-independent SNP were improved in the TP508 (TP) group as compared to the control (CT) group (FIG. 1). Values were significantly different at −8 (p<0.01), −6 (p=0.01) and −5 (p<0.01) in response to ADP, and at −6 (p=0.04) in response to SNP.

Infarct size as a percentage of AAR was 24±4% in the TP508 group as compared to 44±5% in the control group (p=0.01).

Expression of the anti-apoptotic protein Bcl-2 was 2.2-fold higher (p=0.04) in the TP508 group as compared to the control group. Expression of the pro-apoptotic proteins PARP (1.6 fold, p=0.02), cleaved PARP (6.3 fold, p<0.01), and BNIP3 (3.7 fold, p<0.01) were higher in the TP508 group as compared to the control group in the ischemic area. However, the TUNEL+ cell count was increased 1.8 fold (p=0.02) in the AAR in the control group as compared to TP508 group.

This study demonstrated that treatment with a thrombin fragment decreases infarct size and improves endothelial microvascular response in the setting of acute myocardial injury. The expression of Bcl-2 was significantly increased with TP508 treatment, which may account for the reduced number of apoptotic cells. Thus, TP508 may markedly decrease myocardial infarction, improve microvascular function and reduce apoptosis in the myocardiac tissue.

2. The hypercholesterolemic (HC) pigs were fed a high fat diet consisting of 4% cholesterol, 17.2% coconut oil, 2.3% corn oil, 1.5% sodium cholate and 75% regular chow for four weeks. This diet led to an increase in serum cholesterol, LDL and HDL levels from 3.5-9 fold compared to pigs fed a normal diet. The HC pigs underwent the same acute myocardial infarction (AMI) surgical procedure as the normocholesterolemic (NC) pigs as described above and in Section I. Seven pigs (OVC) were normal-cholesterolemic and received vehicle control solution (n=7), Seven pigs (OTC) were normal-cholesterolemic and treated with TP508 as described above (TP, n=7). Seven pigs (OVH) were hyper-cholesterolemic and received vehicle control solution (n=7). Seven pigs (OTH) were hyper-cholesterolemic and treated with TP508 as described above (TP, n=7). Four pigs (OTHF) were hyper-cholesterolemic and treated with a double dose of TP508 (TP, n=4).

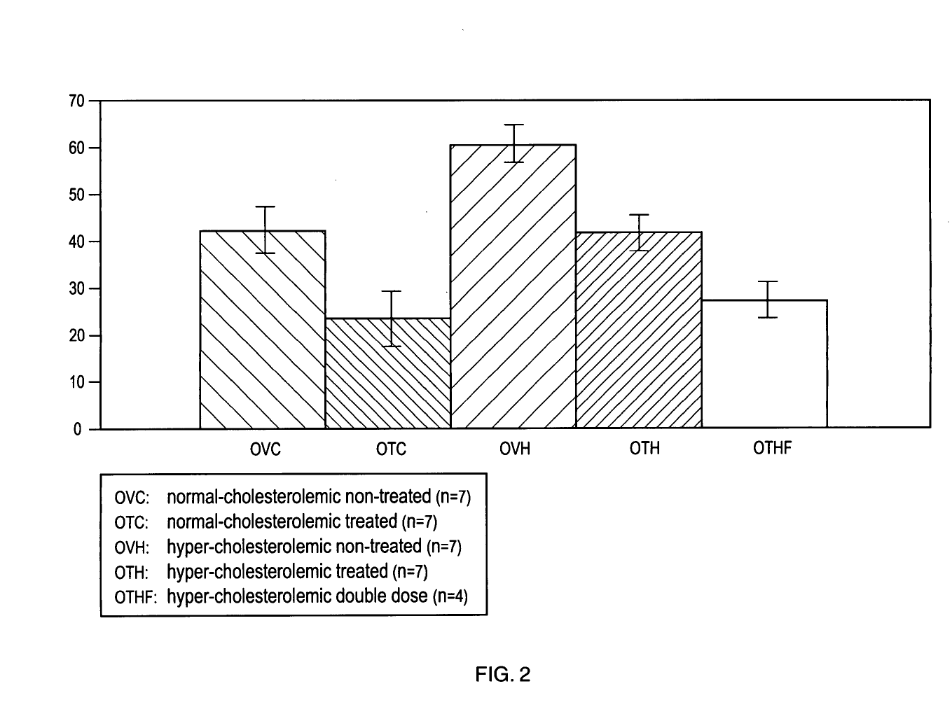

The HC diet lead to a significantly larger infarct compared to the NC pigs (61% of the area at risk (AAR) in HC pigs versus 41% in the NC pigs; p=0.01). As described above in Section I, TP508-treated HC pigs received a bolus dose of 0.5 mg/kg (OTH) or 1.0 mg/kg (OTHF) for 10 minutes prior to reperfusion. This was followed by a slow infusion of 1.25 mg/kg/hr (OTH) or 2.5 mg/kg/hr (OTHF) for the entire two-hour reperfusion period. As shown in FIG. 2, the OTH group had a significantly smaller infarct size compared to the saline-treated OVH control group (40% versus 61%; p=0.003). When the dose of TP508 was doubled in the OTHF group, the infarct size was further reduced to 27% (p=0.003 versus OVH; p=0.01 versus OTH). In summary, the infarct size was significantly decreased in OTC (p=0.03) and OTHF (p=0.0003) as compared to OVH (i.e., from 61% to 40% and to 27%). OVH exhibited approximately 1.5-fold increase in the infarct size as compared to OVC. OTC showed approximately 1.5-fold decrease in the infarct size as compared to OVC.

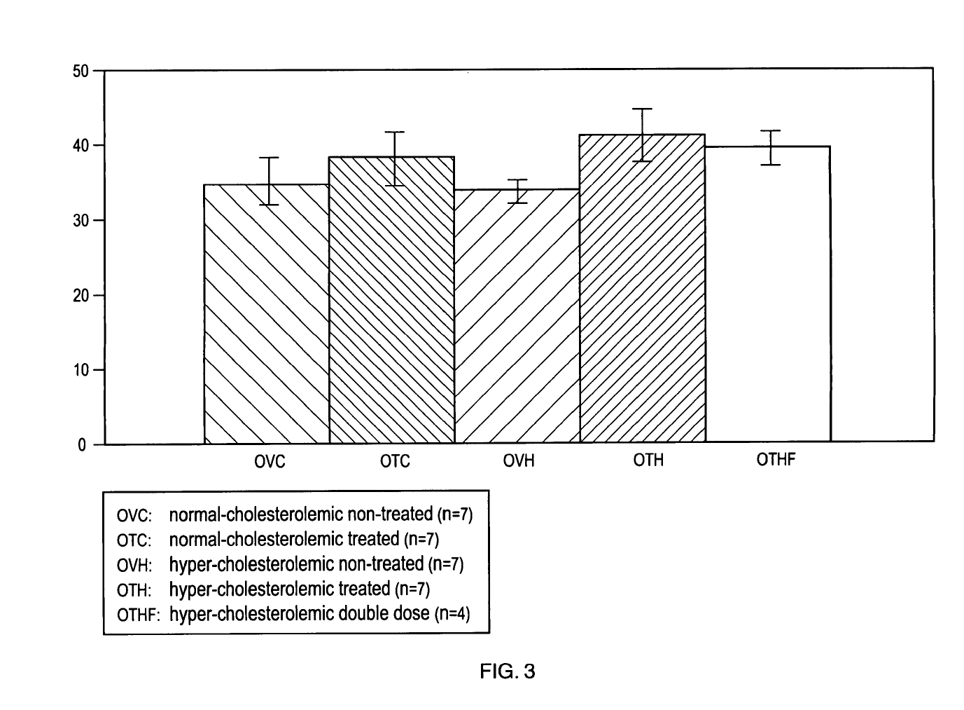

FIG. 3 is a graph showing the area-at-risk (AAR) as a percentage of the total left ventricular mass. FIG. 3 shows that the area-at-risk (AAR) was not significantly different in any of the groups. Seven pigs (OVC) were normal-cholesterolemic and received vehicle control solution (n=7), Seven pigs (OTC) were normal-cholesterolemic and treated with TP508 as described above (TP, n=7). Seven pigs (OVH) were hyper-cholesterolemic and received vehicle control solution (n=7). Seven pigs (OTH) were hyper-cholesterolemic and treated with TP508 as described above (TP, n=7). Four pigs (OTHF) were hyper-cholesterolemic and treated with a double dose of TP508 (TP, n=4) as described above. The infarct area (characterized by absence of staining), the non-infarcted area-at-risk (characterized by red tissue staining), and the non-ischemic portion of the LV (characterized by purple tissue staining) were sharply dissected from one another and weighed. The percentage area-at-risk was defined as: (Infarct mass+non-infarct area-at-risk mass)/Total LV mass×100.

These data demonstrate that TP508 dose-dependently reduces the infarct size in hypercholesterolemic pigs.

### Example 2

### I. Experimental Procedures

To determine the effect of TP508 on infarct size in a normocholesterolemic (NC) porcine model of acute myocardial infarction, NC Yucatan miniswine which weighed approximately 25 kg and were on a normal diet of regular chow were divided randomly into two groups. These groups were: (1) placebo (n=7) and (2) TP508 treatment (n=7). All fourteen (14) animals were subjected to regional left ventricular (LV) ischemia by left anterior descending (LAD) arterial occlusion distal to the second diagonal branch for 60 minutes and to reperfusion for 120 minutes immediately following the arterial occlusion. The two groups received an intravenous (IV) bolus dose of either placebo (NC-control; saline) or 0.5 mg/kg of TP508 (NC-TP508) for 10 minutes prior to the onset of reperfusion. The two groups were subject to a constant IV infusion of placebo (NC-control; saline) or 1.25 mg/kg/hr of TP508 (NC-TP508) for the entire 120-minute reperfusion period.

The experimental procedures described in Example 1 for obtaining and analyzing infarct size in a porcine model were employed in Example 2.

### II. Results

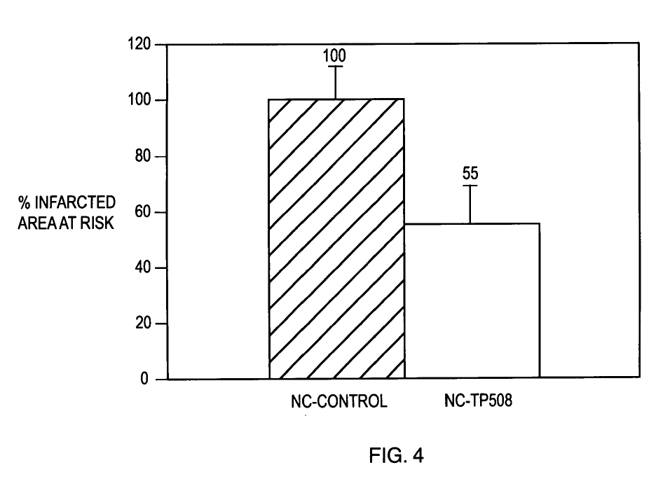

Normal cholesterolemic Yucatan miniswine treated with TP508 (NC-TP508) exhibited significantly smaller infarct sizes as compared to the control group treated with placebo (NC-control). As shown in FIG. 4, treatment with TP508 following an acute ischemic event resulted in significant reduction of infarct size (i.e., 45% reduction as compared to the control group; FIG. 4). This result demonstrates that TP508 has a positive effect on reducing infarct size in the myocardium following acute myocardial infarction.

### Example 3

### I. Experimental Procedures

To determine the effects of TP508 and TP508 dimer on infarct size in a hypercholesterolemic (HC) porcine model of acute myocardial infarction, HC Yucatan miniswine were fed a high cholesterol diet of 4% cholesterol, 17.2% coconut oil, 2.3% corn oil, 1.5% sodium cholate and 75% regular chow, starting at 7 weeks of age and continuing throughout the entire study period. HC Yucatan miniswine weighing approximately 25 kg were divided randomly into five groups, each of which consisted of seven (7) pigs (n=7). These groups were: (1) placebo (HC-Control; n=7); (2) TP508 treatment with an intravenous bolus dose of 0.05 mg/kg and an infusion dose of 0.125 mg/kg/hr (HC-TP508 Dose 0.1×; n=7); (3) TP508 treatment with an intravenous bolus dose of 0.5 mg/kg and an infusion dose of 1.25 mg/kg/hr (HC-TP508 Dose 1×; n=7); (4) TP508 treatment with an intravenous bolus dose of 1.0 mg/kg and an infusion dose of 2.50 mg/kg/hr (HC-TP508 Dose 2×; n=7); and (5) TP508 dimer treatment with a dose equivalent to HC-TP508 Dose 1× on a molar basis (HC-TP508 Dimer; n=7). As described in Examples 1 and 2, all animals were subjected to regional left ventricular (LV) ischemia by left anterior descending (LAD) arterial occlusion distal to the second diagonal branch for 60 minutes followed by reperfusion for 120 minutes. The timing of administration of the doses was the same as in Examples 1 and 2. The same experimental procedures were employed for obtaining and analyzing infarct size as described in detail in Example 1.

### II. Results

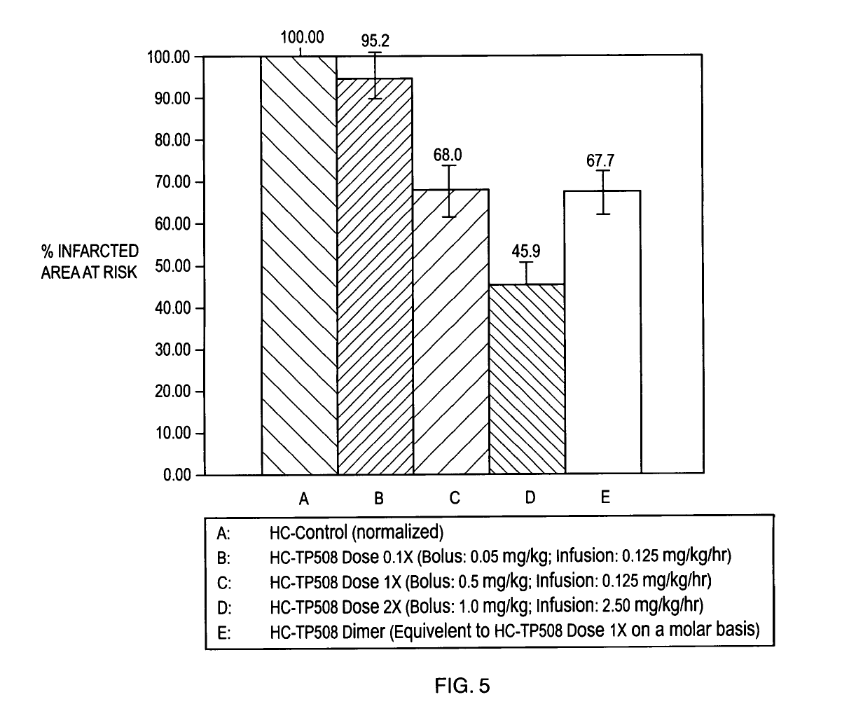

As shown in FIG. 5, hypercholesterolemic (HC) Yucatan miniswine treated with TP508 (HC-TP508 Dose 0.1×; HC-TP508 Dose IX; and HC-TP508 Dose 2×) or TP508 dimer (HC-TP508 Dimer) exhibited smaller infarct sizes as compared to the control group treated with placebo (HC-control). Treatment with TP508 following an acute ischemic event resulted in significant reduction of infarct size: 32% reduction in the HC-TP508 Dose 1× group and 54% reduction in the HC-TP508 Dose 2× group as compared to the control group (FIG. 4). Furthermore, similar to TP508, TP508 dimer also showed a positive effect on reducing infarct size. HC Yucatan miniswine treated with TP508 dimer at a molar dose equivalent to the dose used for the HC-TP508 Dose 1× group exhibited 32% reduction in infarct size, compared to control, a reduction similar to that observed in the HC-TP508 Dose 1× group.

These results demonstrate that both TP508 and TP508 dimer have positive effects on reducing infarct size in the myocardium following acute myocardial infarction.

While this invention has been particularly shown and described with references to example embodiments thereof, it is understood by those skilled in the art that various changes in form and details may be made therein without departing from the scope of the invention encompassed by the appended claims.

1. A method of treating acute myocardial infarction in a subject, the method comprising administering to the subject a therapeutically effective amount of an agonist of a non-proteolytically activated thrombin receptor during the acute myocardial infarction.

2-3. (canceled)

4. The method of claim 52, wherein the agonist is a thrombin peptide derivative comprising the amino acid sequence Asp-Ala-R, wherein R is a serine esterase conserved sequence, and wherein the thrombin peptide derivative is about 12 to about 23 amino acid residues in length.

5-11. (canceled)

12. The method of claim 4, wherein the thrombin peptide derivative comprises an N-terminus which is unsubstituted, and a C-terminus which is unsubstituted or a C-terminal amide represented by —C(O)NH₂.

13-17. (canceled)

18. The method of claim 12, wherein the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative having at least eighteen amino acid residues, wherein X₁ is Glu or Gln and X₂ is Phe, Met, Leu, His or Val.

19. A method of treating an acute myocardial infarction in a subject, said method comprising administering to the subject a therapeutically effective amount of an agonist of a non-proteolytically activated thrombin receptor, wherein the agonist is the polypeptide Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

20-28. (canceled)

29. The method of claim 12, wherein the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5) or a fragment thereof comprising amino acid residues 10-18 of SEQ ID NO:5, wherein Xaa is alanine, glycine, serine or an S-protected cysteine; X₁ is Glu or Gln; and X₂ is Phe, Met, Leu, His or Val.

30-34. (canceled)

35. The method of claim 52, wherein the agonist is a peptide dimer comprising two thrombin peptide derivatives 12 to 23 amino acid residues in length which, independently, comprise the polypeptide Asp-Ala-Cys-X₁-Gly-Asp-Ser-Gly-Gly-Pro-X₂-Val (SEQ ID NO:10), wherein X₁ is Glu or Gln and X₂ is Phe, Met, Leu, His or Val,
the dimer is essentially free of monomer;
the thrombin peptide derivatives are the same; and
the thrombin peptide derivatives are covalently linked through a disulfide bond.

36-40. (canceled)

41. The method of claim 35, wherein the thrombin peptide derivatives each comprise an N-terminus which is unsubstituted; and a C-terminus which is unsubstituted or a C-terminal amide represented by —C(O)NH₂.

42-45. (canceled)

46. The method of claim 41, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val or a fragment thereof comprising amino acid residues 10-18 of SEQ ID NO:2.

47. The method of claim 41, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val.

48-51. (canceled)

52. A method of reducing apoptosis of myocardial tissue in a subject undergoing acute myocardial infarction, the method comprising administering to the subject a therapeutically effective amount of an agonist of a non-proteolytically activated thrombin receptor during the acute myocardial infarction.

53. The method according to claim 52, wherein the agonist is a peptide dimmer (SEQ ID NO. 3) represented by the following structural formula:

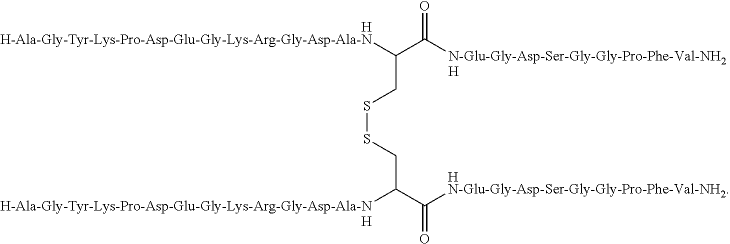

54. The method according to claim 52, wherein the agonist is administered within 120 minutes of an onset of myocardial infarction.

55. The method according to claim 52, wherein the agonist is administered within 6 hours of an onset of myocardial infarction.

56. The method according to claim 52, wherein the agonist is administered within 7 days of an onset of myocardial infarction.

57. The method according to claim 52, wherein the acute myocardial infarction occurs in the left ventricular wall.

58. The method according to claim 52, wherein the acute myocardial infarction occurs in the right ventricular wall.

59-61. (canceled)

62. The method according to claim 52, wherein the agonist is the polypeptide H-Ala-Gly-Tyr-Lys-Pro-Asp-Glu-Gly-Lys-Arg-Gly-Asp-Ala-Ser-Glu-Gly-Asp-Ser-Gly-Gly-Pro-Phe-Val-NH₂ (SEQ ID NO:28).

63. The method of claim 52, wherein said reduction of apoptosis of myocardial tissue in a subject is determined by lower levels of one or more protein factors associated with apoptosis compared to untreated control.

64. The method of claim 63, wherein said protein factors are selected from the group consisting of apoptosis inducing factors (AIF), bad, and cleaved-caspase 3.

65. The method according to claim 1, wherein the agonist is a peptide dimer represented by the following structural formula:

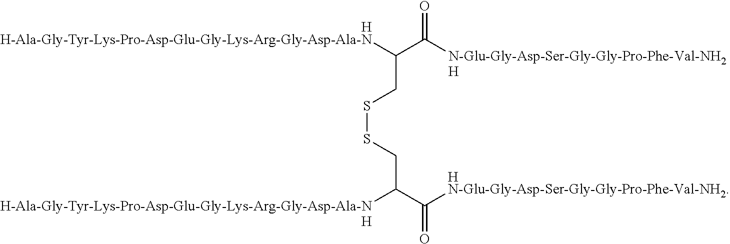

66. The method according to claim 1, wherein the agonist is administered within 120 minutes of an onset of myocardial infarction.

67. The method according to claim 1, wherein the agonist is administered within 6 hours of an onset of myocardial infarction.
